(19) United States
(12) Patent Application Publication
(10) Pub. No.: US 2014/0323815 A1
(43) Pub. Date: Oct. 30, 2014

(54) APPARATUS FOR CONTROLLING MEDICAL DEVICE SAFETY AND METHOD FOR SAME

(71) Applicant: NOHSN CO., LTD, Iksan-si (KR)

(72) Inventor: Cheol-Min HAN, Jeonju-si (KR)

(73) Assignee: NOHSN CO., LTD, Iksan-si (KR)

(21) Appl. No.: 14/289,666

(22) Filed: May 29, 2014

Related U.S. Application Data
(63) Continuation of application No. PCT/KR2013/004884, filed on Jun. 3, 2013.

(30) Foreign Application Priority Data
Jun. 1, 2012 (KR) 10-2012-0059307

(51) Int. Cl. A61B 5/00

(52) U.S. Cl. CPC A61B 5/72; A61B 5/7405
USPC 600/300

(57) ABSTRACT

There are provided a device and method for controlling medical device safety. According to the present invention, the device for controlling medical device safety monitors a patient's body state information in real time, predicts a risk state caused by an amount of a body change of the patient by monitored body state information or combinations thereof, and controls a medical device in the event of risky situations.

### TECHNICAL FIELD

Embodiments of the present invention relate to technology for diagnosis and/or treatment in the field of medicine, and more specifically, to body diagnosis and monitoring technology for diagnosing and monitoring body states of patients, bio signal processing technology, and technology for controlling medical devices including diagnostic and/or treatment devices.

### BACKGROUND ART

Through all ages, the importance of medicine has been emphasized. Various industries related to medicine are rapidly under development based on human-centered thinking. In terms of technological aspects of medicine, due to rapid development of technology and theory, medical devices capable of accurately and precisely diagnosing have been developed. According to the development of the medical devices, remarkable progress has been made in diagnosis of human diseases. Medical devices for diagnosing and treating human diseases are developing from day to day.

However, in the field of medicine, various types of medical accidents frequently occur during common procedures. In particular, when operators are unable to quickly handle risky situations occurring during procedures using medical devices, it is likely to be directly linked to medical accidents.

### DISCLOSURE

### Technical Problem

According to an embodiment, there are provided a device and method for controlling medical device safety capable of safely controlling a medical device.

### Technical Solution

According to an aspect of the present invention, there is provided a device for controlling medical device safety. The device includes a body state information obtaining unit configured to obtain body state information from at least one diagnostic device for diagnosing a body; a data processing unit configured to extract an identifier from the body state information obtained by the body state information obtaining unit, extract the body state information corresponding to the extracted identifier, and classify the extracted body state information for each identifier; a risk estimating unit configured to generate risk estimation information according to a body state change based on the body state information extracted or classified by the data processing unit; a risk state determining unit configured to determine a risk state using the risk estimation information of the risk estimating unit; and a medical device control unit configured to control an external device including a medical device for diagnosis and treatment according to the risk state determination result of the risk state determining unit.

The identifier may include at least one among a user identifier, a diagnostic device identifier, a time identifier, a service flow identifier, and a priority identifier according to the diagnostic device identifier or the service flow identifier.

When the body state information obtained by the body state information obtaining unit does not include the identifier, the data processing unit may assign the identifier to the obtained body state information, extract the body state information to which the identifier is assigned, and classify the extracted body state information for each identifier.

The risk estimation information generated by the risk estimating unit may have a form of a management table of the body state information for each parameter, and the management table for each parameter may include information on a risk estimation group, a level rank, a volume size, and a risk level.

The risk determining unit may determine a risk state using a risk estimation value that is obtained by calculating body state information having no weight or having an assigned weight for each parameter within the same time range or a predetermined time range.

The device may further include a weight assigning unit configured to assign a weight to the body state information extracted or classified by the data processing unit for each service flow, wherein the risk estimating unit may generate risk estimation information from the body state information to which the weight is assigned by the weight assigning unit.

The device may further include an alarming unit configured to receive the risk state determination result from the risk state determining unit through the medical device control unit and alarm the outside to the result when the risk is estimated based on the risk state determination result of the risk state determining unit.

The device may further include a body state information managing unit configured to receive the body state information classified by the data processing unit, put the information into a database along with a regular value range, statistically calculate the body state information according to a body state information management setting value and a service flow, transmit a statistical result to the risk estimating unit, receive a risk estimation result from the risk estimating unit, and externally output the result. In this case, the body state information management setting value may include at least one among an identifier, a volume level, a risk level, a time, a risk estimation group, a volume size, and a level rank, and the service flow may include at least one among a body type, a physical constitution, disease and a family history.

The device may further include a risk information managing unit configured to manage risk information including a normal range and a risk state range, manage and put information on a risk state list, a current risk state, and an estimated risk state into a database within a risk level rank range according to risk estimation of the risk estimating unit.

The device may further include a data comparison unit configured to compare the risk estimation information generated by the risk estimating unit with a body state information reference value stored in the risk information managing unit, wherein the risk state determining unit may determine the risk state using a comparison result of the data comparison unit.

### Advantageous Effects

According to the embodiment, it is possible to monitor a patient's body state information in real time, previously estimate a risk state caused by an amount of a body change of the patient by monitored body state information or combinations thereof, and control a medical device in the event of risky situations. Accordingly, it is possible to perform control such that the medical device safely performs operations without a procedure depending on operators. Furthermore, it is possible to prevent medical accidents frequently caused by procedural methods dependent on the know-how of the operators, and operations of the medical device may be controlled to perform safer procedures.

Moreover, the present invention may be used in a system in which a diagnostic device and a treatment device are integrated, provide a safer procedural guideline than a current system having a structure in which a patient monitoring device, a diagnostic device, and a treatment device independently operate, and is able to be used for a one stop automation system that may diagnose and treat at the same time.

### DESCRIPTION OF DRAWINGS

FIG. 1 is a diagram illustrating a configuration of a system for controlling medical device safety according to an embodiment of the present invention;

FIG. 2 is a diagram illustrating a configuration of a device for controlling medical device safety according to an embodiment of the present invention;

FIG. 3 is a reference diagram illustrating an interface provided for obtaining body state information of a body state information obtaining unit according to an embodiment of the present invention;

FIG. 4 is a reference diagram illustrating a process of a data processing unit assigning and classifying an identifier according to an embodiment of the present invention;

FIG. 5 is a diagram illustrating an identifier management structure of a data processing unit according to an embodiment of the present invention;

FIG. 6 is a diagram illustrating a risk level management structure of a risk estimating unit according to an embodiment of the present invention;

FIG. 7 is a reference diagram illustrating exemplary parameter value management and setting of a function setting unit according to an embodiment of the present invention;

FIG. 8 is a diagram illustrating a structure of a management table for each parameter generated by the risk estimating unit according to an embodiment of the present invention;

FIG. 9 is a diagram illustrating a structure of a management table of a blood pressure parameter which is one of the parameters according to an embodiment of the present invention;

FIG. 10 is a diagram illustrating an exemplary weight range for each service flow of a weight assigning unit according to an embodiment of the present invention;

FIG. 11 is a diagram illustrating a structure of a body state information database of a body state information managing unit according to an embodiment of the present invention;

FIG. 12 is a diagram illustrating a structure of a regular value range database according to body state information of the body state information managing unit according to an embodiment of the present invention;

FIG. 13 is a diagram illustrating a structure of the body state information database according to a time of the body state information managing unit according to an embodiment of the present invention;

FIG. 14 is a diagram illustrating a structure of a statistical body state information database of the body state information managing unit according to an embodiment of the present invention;

FIG. 15 is a diagram illustrating a structure of a risk information table put into a database by a risk information managing unit according to an embodiment of the present invention; and

FIG. 16 is a flowchart illustrating a method of controlling medical device safety of the device for controlling medical device safety according to an embodiment of the present invention.

### DETAILED DESCRIPTIONS

While the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention.

In descriptions of the invention, when it is determined that detailed descriptions of related well-known technology unnecessarily obscure the gist of the invention, the detailed descriptions thereof will be omitted. The numbers or symbols used in description of the specification are used only to distinguish one component from another.

In the specification, it will be understood that when an element is referred to as being “connected” or “coupled” to another element, it can be directly connected or coupled to the other element or intervening elements may be present unless the context clearly indicates otherwise.

Hereinafter, embodiments of the invention will be described in detail with reference to the accompanying drawings. In description of the invention, in order to facilitate overall understanding, like reference numerals are used for like elements even in different drawings.

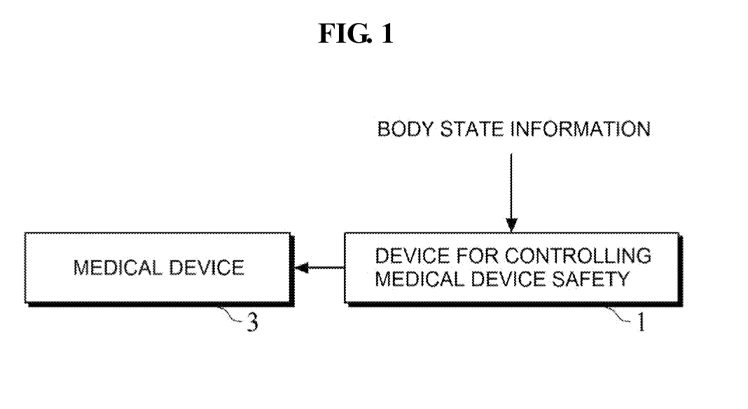

FIG. 1 is a diagram illustrating a configuration of a system for controlling medical device safety according to an embodiment of the present invention.

As illustrated in FIG. 1, a device for controlling medical device safety 1 according to an embodiment of the present invention obtains a patient's body state information, estimates the patient's risk state using the obtained body state information, controls a medical device 3 based on risk estimation information, or alarms the outside to risky situations.

The body state information of the patient has a form of a numerical value measured for each parameter such as blood glucose, blood pressure, an electrocardiogram, and body fat. The body state information of the patient may be obtained from a diagnostic device configured to diagnose the patient's body. Examples of the diagnostic device may include a blood glucose meter, a sphygmomanometer, an electrocardiogram device, and a body fat analyzer. The medical device 3 is a device that diagnoses and/or treats the patient under control of the device for controlling medical device safety 1.

It is possible to safely protect the patient from risky states that may occur in the field of medicine using the device for controlling medical device safety 1 of the present invention. For example, it is possible to predict emergency situations caused by an amount of a body change of the patient during a procedure or it is possible to previously prevent risky situations caused by a control delay of the medical device 3.

According to an additional embodiment, the device for controlling medical device safety 1 combines body state information obtained in real time with previous body state information and puts the combined information into a database. Also, risk estimation information in which the patient's risky situations are estimated may be put into a database and managed.

A configuration and a process of the device for controlling medical device safety 1 having the above-described features will be described in detail with reference to the accompanying drawings to be described.

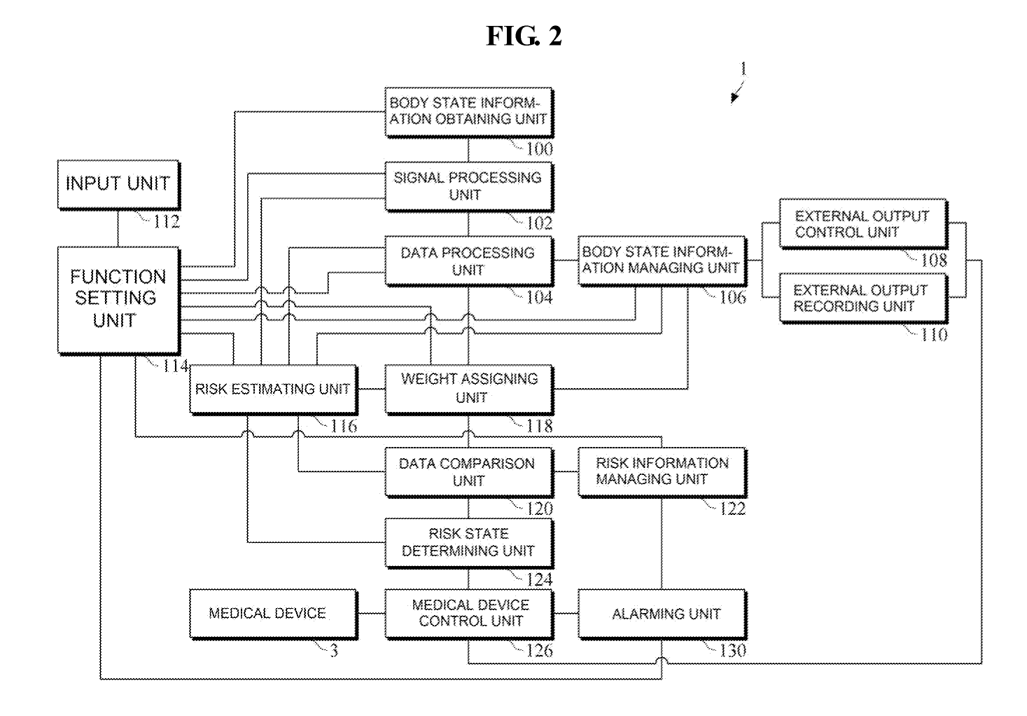

FIG. 2 is a diagram illustrating a configuration of the device for controlling medical device safety 1 according to an embodiment of the present invention.

FIG. 2 is an exemplary configuration of performing a safety control function of the device for controlling medical device safety 1 of the present invention and thus components may be added, deleted, and changed.

As illustrated in FIG. 2, the device for controlling medical device safety 1 includes a body state information obtaining unit 100, a data processing unit 104, a risk estimating unit 116, a risk state determining unit 124, and a medical device control unit 126, and may further include a signal processing unit 102, a body state information managing unit 106, an external output control unit 108, an external output recording unit 110, an input unit 112, a function setting unit 114, a weight assigning unit 118, a data comparison unit 120, a risk information managing unit 122, and an alarming unit 130.

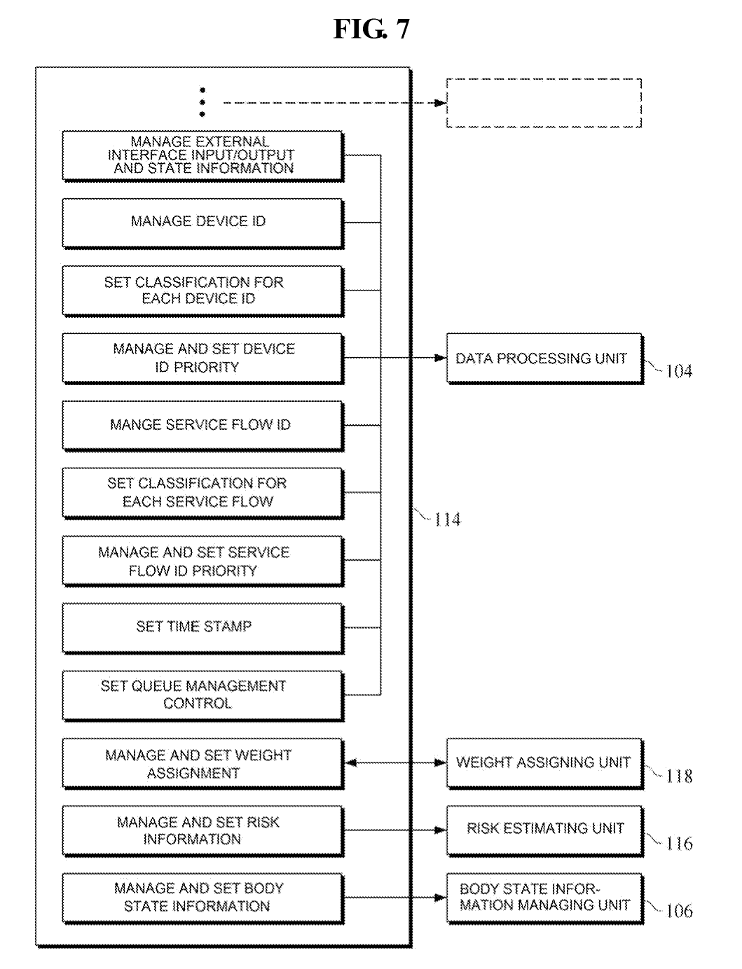

The function setting unit 114 manages and sets parameter values necessary for performing operations of other components, for example, manages external interface input/output and state information, and manages and sets various identifiers. An embodiment in which the function setting unit 114 manages and sets parameter values will be described in detail below with reference to FIG. 7. The input unit 112 performs a function of assigning or instructing each function setting value to the function setting unit 114.

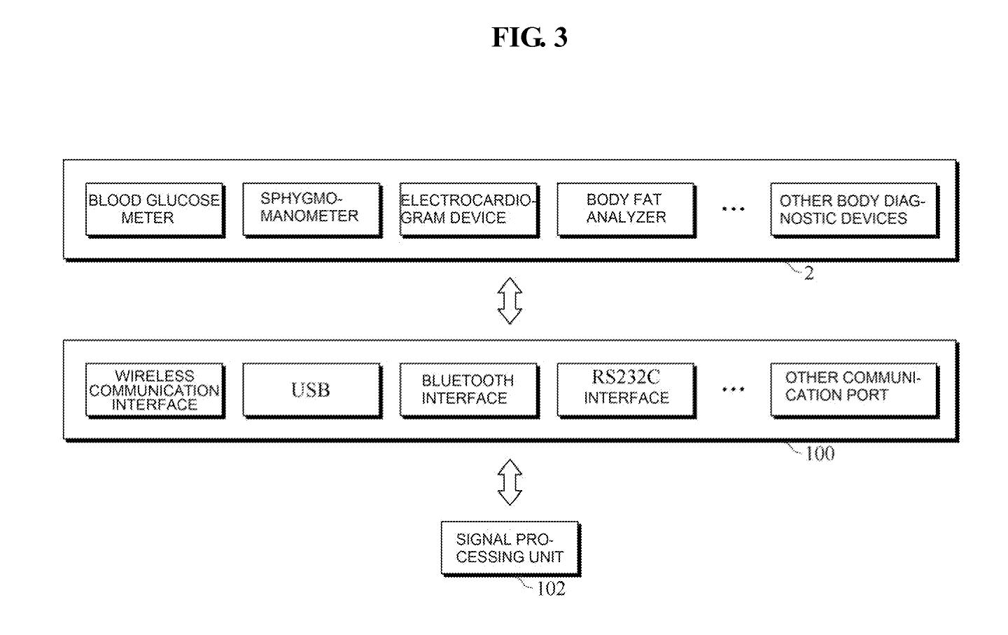

The body state information obtaining unit 100 obtains body state information from diagnostic devices configured to diagnose the patient's body according to the setting values of the function setting unit 114. Examples of the diagnostic device may include a blood glucose meter, a sphygmomanometer, an electrocardiogram device, and a body fat analyzer. The body state information has a form of a numerical value measured for each parameter such as blood glucose, blood pressure, an electrocardiogram, and body fat. The body state information obtaining unit 100 may obtain body measurement information measured by the diagnostic device in real time. In addition, it is possible to obtain the body state information using various user interfaces, and an embodiment thereof will be described below with reference to FIG. 3.

The signal processing unit 102 converts the information obtained by the body state information obtaining unit 100 into a form that can be calculated according to the setting value of the function setting unit 114. An independent signal line is assigned to transmit risk information and transmits the risk information to the risk estimating unit 116.

The data processing unit 104 receives the body state information obtained by the body state information obtaining unit 100 from the signal processing unit 102, and extracts a management code or identifier (ID) information for each user from the received body state information. Then, the body state information corresponding to the extracted identifier is extracted and the extracted body state information for each identifier is classified according to the setting value of the function setting unit 114.

When the body state information obtained by the body state information obtaining unit 100 does not include the identifier, the identifier is assigned to the obtained body state information, and then the body state information to which the identifier is assigned may be extracted. Then, the extracted body state information for each identifier may be classified according to the setting value of the function setting unit 114. The data processing unit 104 may deliver the body state information classified for each identifier to the body state information managing unit 106, the risk estimating unit 116, and the weight assigning unit 118.

The identifier includes a user identifier, a device identifier, a time identifier, a service flow identifier according to the device identifier, and a priority identifier according to the device or the service flow identifier. In this case, a service flow is a parameter capable of determining a body condition, for example, the patient's blood pressure, blood glucose, weight, height, and body temperature. In the body state information to which the device identifier is assigned, one piece of body state information obtained from a single diagnostic device may be assigned and several pieces of body state information obtained from a single diagnostic device may be assigned.

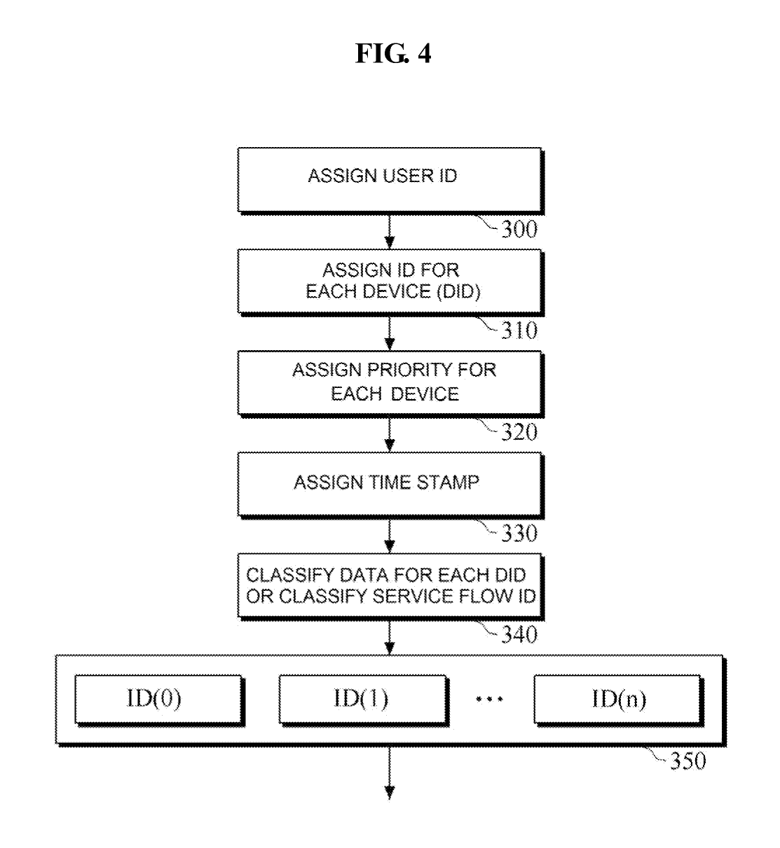

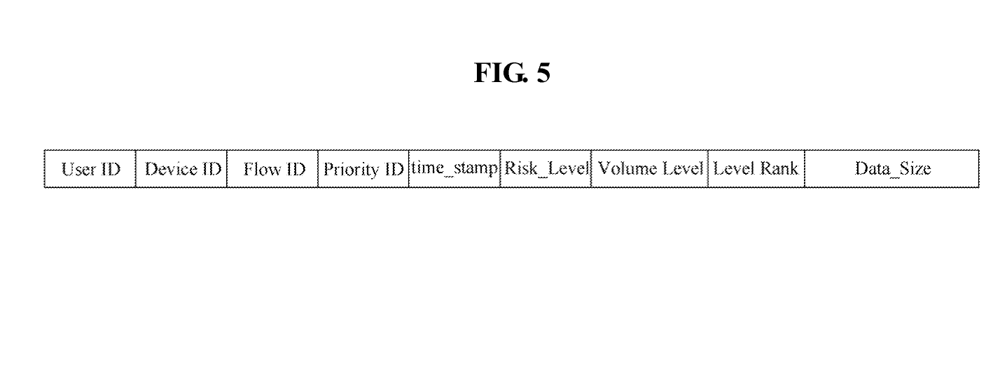

According to an embodiment, the data processing unit 104 creates a new identifier according to the user's command or setting of the function setting unit 114 and assigns the created identifier to the body state information. Also, the data processing unit 104 may classify and manage each identifier according to diagnosis and/or procedure setting values of the user or the function setting unit 114. An embodiment of a process of the data processing unit 104 assigning and classifying an identifier will be described below with reference to FIG. 4. An embodiment of an identifier management structure of the data processing unit 104 will be described below with reference to FIG. 5.

The body state information managing unit 106 manages the body state information according to a body state information management function setting value of the function setting unit 114. The body state information management function setting value includes information on an identifier, a volume level, a risk level, a time stamp, a risk estimation group (RE_Group), a volume size, a level rank, and high_low. In this case, the body state information management function setting value may be added and managed in order to effectively determine the body's risk state according to the patient's disease and body state.

According to an embodiment, the body state information managing unit 106 receives the body state information classified by the data processing unit 104, puts the body state information for each service flow according to the body state information management function setting value of the function setting unit 114 into a database, and may provide the database to the risk estimating unit 116. In this case, the body state information put into the database may have a form of statistical information.

According to an embodiment, the body state information managing unit 106 receives a risk estimation result from the risk estimating unit 116 and transmits the result to the external output control unit 108 and/or the external output recording unit 110. In this case, in order to adjust estimation accuracy, the risk estimating unit 116 requests a change of the body state information management function setting value from the function setting unit 114 or the body state information managing unit 106, and may repeatedly perform risk estimation according to the changed function setting value.

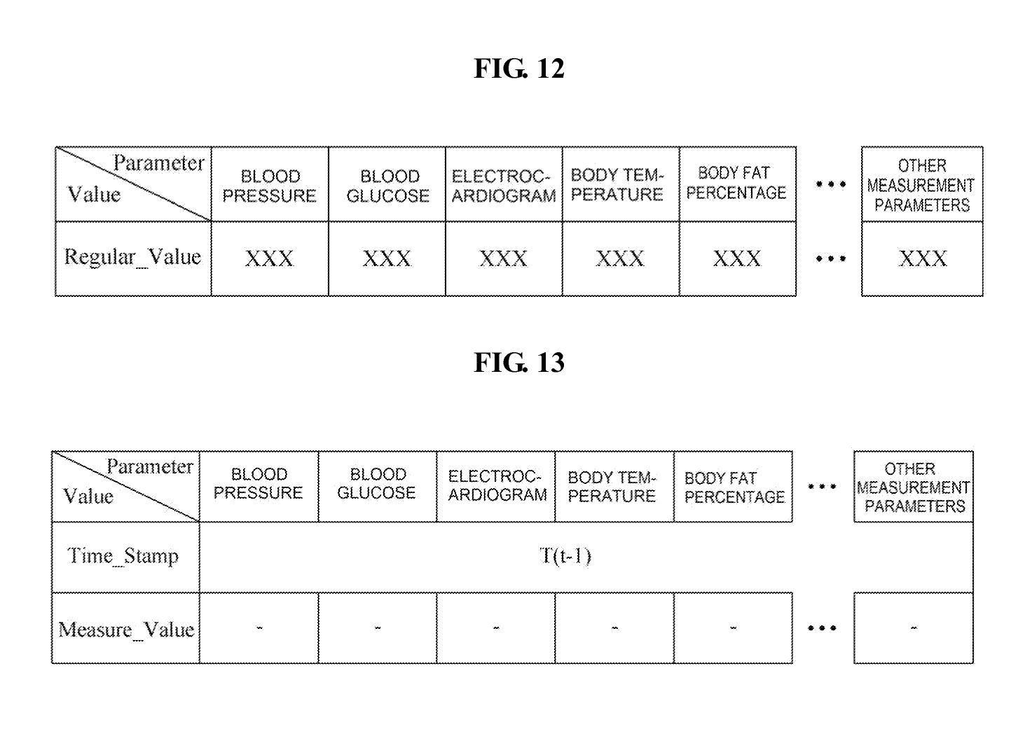

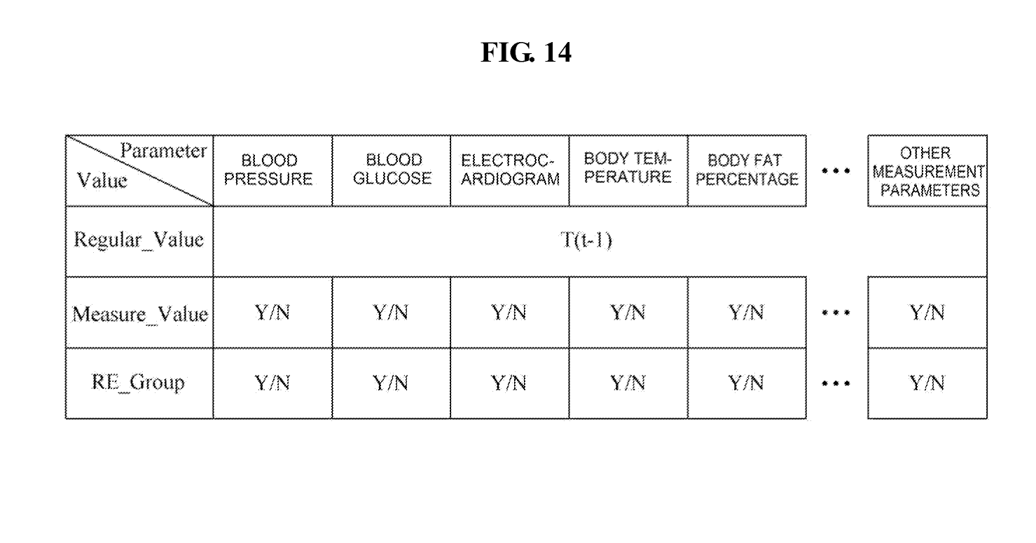

An embodiment of a structure of the body state information database of the body state information managing unit 106 will be described below with reference to FIG. 11. An embodiment of a structure of a regular value range database according to the body state information of the body state information managing unit 106 will be described below with reference to FIG. 12. An embodiment of a structure of the body state information database according to a time of the body state information managing unit 106 will be described below with reference to FIG. 13. An embodiment of a structure of the statistical body state information database of the body state information managing unit 106 will be described below with reference to FIG. 14.

The external output control unit 108 controls the diagnostic device for obtaining the body state information, a medical device for diagnosis and/or treatment, and devices for sharing the obtained body state information. The devices are external devices that are previously registered by the function setting unit 114. In this case, the external output control unit 108 may control operations of the external devices or provide risk state information according to the body state information obtained by the body state information managing unit 106 and the risk estimation information generated by the risk estimating unit 116. Moreover, the external output control unit 108 may be synchronized with the external devices in order to control the external devices.

The external output recording unit 110 records and manages a history of providing the body state information and the patient's risk estimation information to the external devices or managing operations thereof. In this case, the recorded history may be used to analyze a control performance result of the external device.

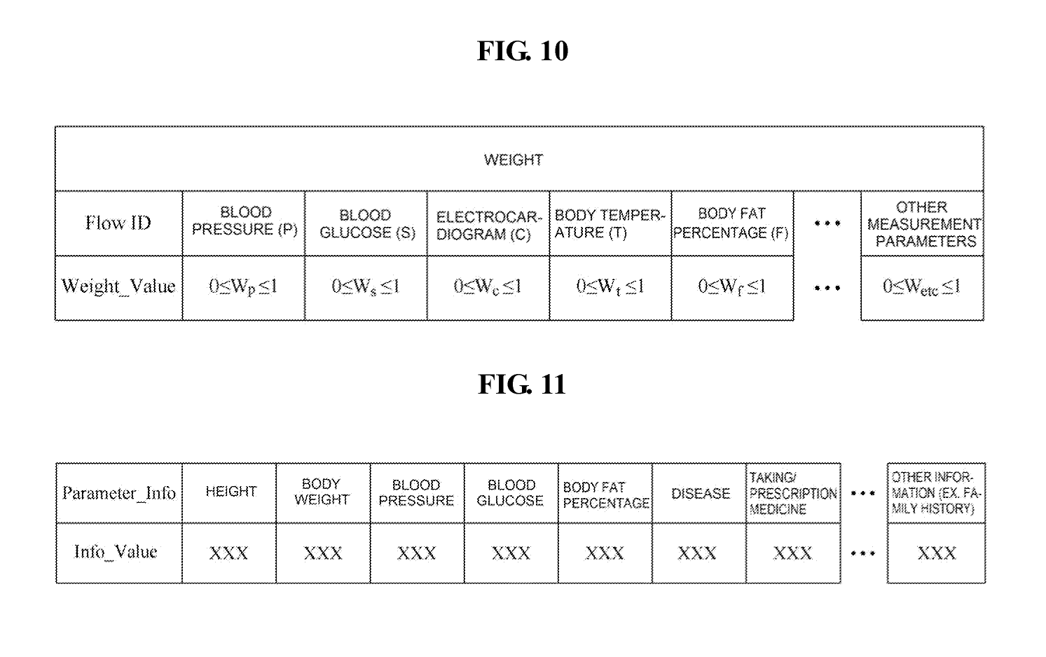

The weight assigning unit 118 assigns a weight to the body state information extracted or classified by the data processing unit 104 for each service flow. In this case, the weight assigning unit 118 may assign a weight according to a weight assignment value set based on each service flow by the function setting unit 114. A reference for setting the weight assignment value may vary according to an initial value set based on each disease or the body state for each service flow according to the body state information of the patient or a user setting value. The body state information of the patient includes the body state information obtained by the body state information obtaining unit 100, a change amount of each service flow according to a time, a family history, a disease history, and the like. An example of the weight assigning unit 118 assigning a weight for each service flow will be described below with reference to FIG. 10.

A process of the weight assigning unit 118 assigning a weight will be described. The weight assigning unit 118 receives a body state information service flow risk level from the function setting unit 114. In this case, when Enable is set to “1,” a weight value of the service flow identifier is loaded, the weight is assigned to a corresponding service flow, and the assigned weight is calculated with data, and then output. On the other hand, when Enable is not “1,” data is directly output.

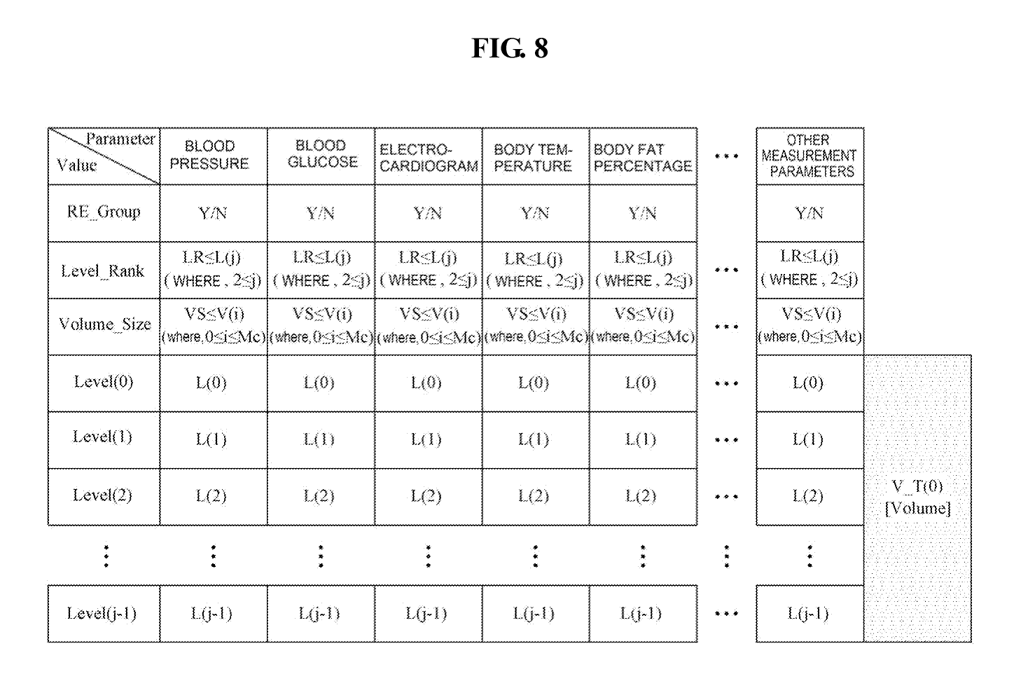

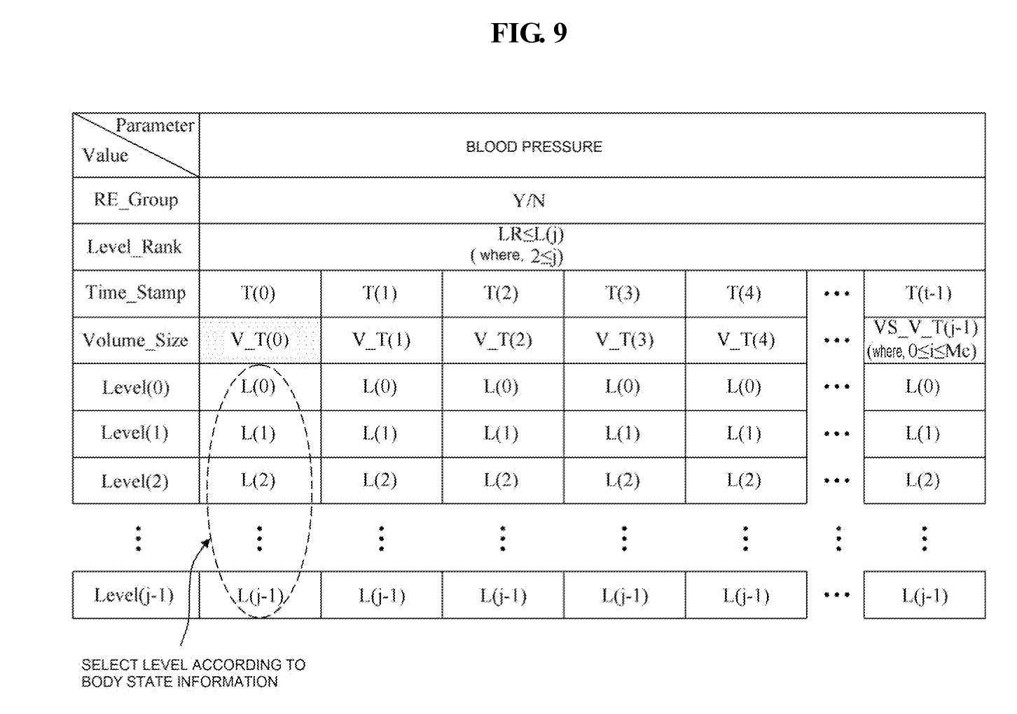

The risk estimating unit 116 generates risk estimation information according to a body state change based on the body state information classified by the data processing unit 104. The risk estimation information may have a form of a management table of the body state information for each parameter. Here, examples of the parameter include blood pressure, blood glucose, an electrocardiogram, a body temperature, and a body fat percentage. The management table for each parameter may include fields of a risk estimation group (RE_Group), a level rank, a volume size, and a risk level. An embodiment of the management table for each parameter generated by the risk estimating unit 116 will be described below with reference to FIGS. 8 and 9.

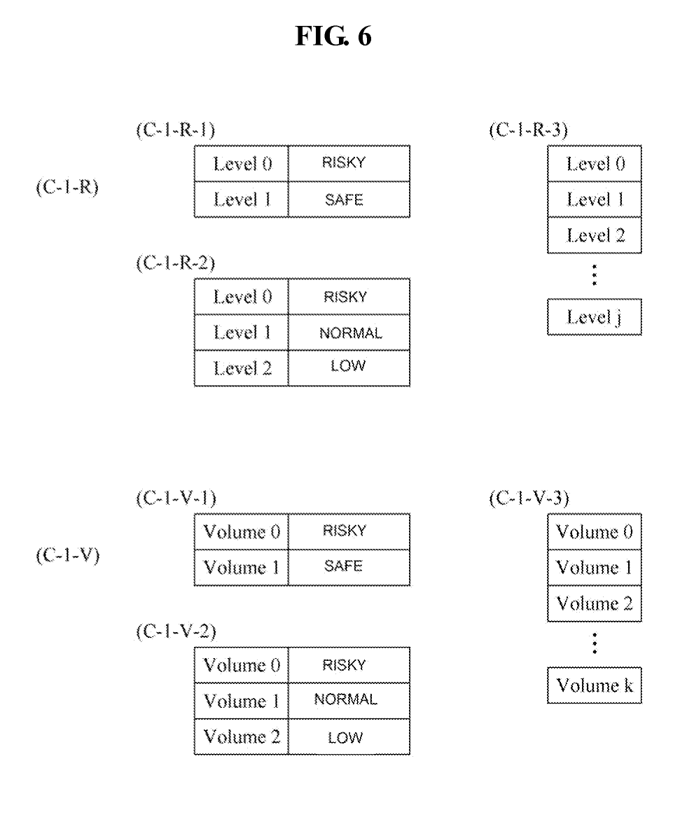

Risk level steps for managing a risk level of the risk estimating unit 116 may be classified as C-1-R-1 (risk level 1), C-1-R-2 (risk level 2), C-1-R-3 (risk level 3), C-1-V-1 (volume level 1), C-1-V-2 (volume level 2), or C-1-V-3 (volume level 3). A risk level management structure of the risk estimating unit 116 will be described below with reference to FIG. 6.

The data comparison unit 120 compares the risk estimation information generated by the risk estimating unit 116 with a body state information reference value stored in the risk information managing unit 122. The risk estimation information may be information to which the weight assigned by the weight assigning unit 118 is reflected by a weight setting value of the function setting unit 114. The data comparison unit 120 delivers a comparison result value to the risk state determining unit 124 in order to determine the risk state.

The risk information managing unit 122 manages risk information according to the risk information setting value set by the function setting unit 114. The risk information includes the body state information measurement reference value, and may include a normal range and risk state range value for each service flow.

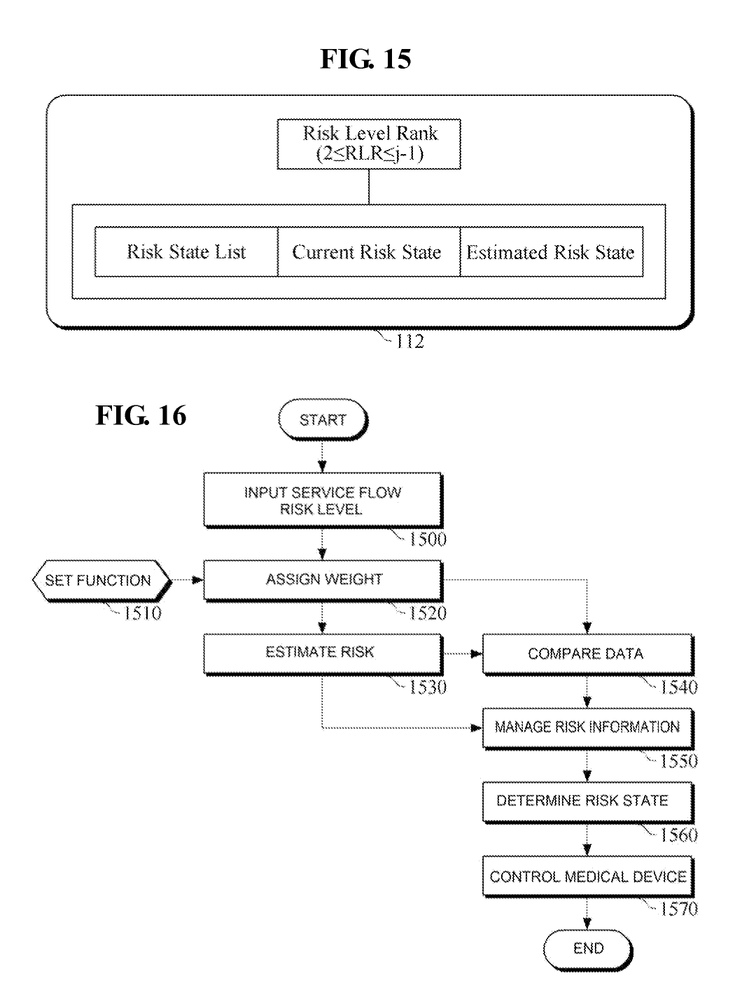

According to an embodiment, the risk information managing unit 122 places and manages information on a risk state list, a current risk state, and an estimated risk state within a risk level rank range according to the risk estimation of the risk estimating unit 116 into a database. That is, the risk information managing unit 122 records current and estimated risk state information of risk state lists generated according to the patient's body state change in a risk information table. Then, the risk state information is delivered to the alarming unit 130. An embodiment of the risk information table put into a database by the risk information managing unit 122 will be described below with reference to FIG. 15.

The risk state determining unit 124 determines the risk state using the risk estimation information of the risk estimating unit 116. In this case, the risk state determining unit 124 may determine the risk state using the comparison result of the data comparison unit 120. Risk state determination information is delivered to the medical device control unit 126 and is used for the medical device control unit 126 to control the external devices including the medical device 3.

According to the present invention, the risk state determining unit 124 may determine the risk state using various methods.

According to an embodiment, the risk state determining unit 124 may determine the risk state by Equation 1.

$$Thresholdvalue \leq {\sum }_{L = 0}^{L = j} [ P ( j ) + S ( j ) + C ( j ) + T ( j ) + F ( j ) … ] \qquad [ Equation 1 ]$$

In Equation 1, a risk estimation value is a value of a sum of body state information for each parameter in the same time (Time_Stamp) zone without weight. In Equation 1, each parameter includes blood pressure (P), blood glucose (S), an electrocardiogram (C), a body temperature (T), a body fat percentage (F), and the like. L denotes a risk state. When a risk level rank is 5, L has a value between 0 and 5. In this case, the risk state determining unit 124 may determine the risk state, for example, risky, safe, and cautious states, using the risk estimation value calculated by Equation 1.

According to another embodiment, the risk state determining unit 124 may determine the risk state by Equation 2.

$$Thresholdvalue \leq {\sum }_{L = 0}^{L = j} [ P ( j ) * {W}_{p} + S ( j ) * {W}_{s} + C ( j ) * {W}_{c} + T ( j ) * {W}_{t} + F ( j ) * {W}_{f} … ] \qquad [ Equation 2 ]$$

In Equation 2, a risk estimation value is a value of a sum of body state information to which different weights (W) are assigned for each parameter in the same time (Time_Stamp) zone. Wp denotes a weight of blood pressure (P), Ws denotes a weight of blood glucose (S), We denotes a weight of an electrocardiogram (C), Wt denotes a weight of a body temperature (T), and Wf denotes a weight of body fat percentage (F). L denotes the risk state. When the risk level rank is 5, L has a value between 0 and 5. In this case, the risk state determining unit 124 may determine the risk state, for example, risky, safe, and cautious states, using the risk estimation value calculated by Equation 2.

Alternatively, in addition to the calculation by the above-described Equations, the risk state may be determined by various methods. For example, a value of a sum of body state information corresponding to a time stamp (Time_Stamp) range according to a volume size value without weight may be used as the risk estimation value.

The medical device control unit 126 controls the medical device 3 for diagnosis and treatment and/or the alarming unit 130 according to the risk state determination result of the risk state determining unit 124. The medical device 3 is a device for diagnosing and/or treating the patient under control of the medical device control unit 126. The medical device control unit 126 may control the medical device 3 in the event of emergency situations caused by an amount of a body change of the patient during a procedure in the field of medicine. In addition, in the event of emergency situations, the emergency situations are externally output through the alarming unit 130 such that a doctor may handle risky situations.

When the risk is estimated based on the risk state determination result of the risk state determining unit 124, the alarming unit 130 receives the risk state determination result from the risk state determining unit 124 through the medical device control unit 126 and alarms the outside to the result. As a type of alarm, all types of alarm methods may be used, for example, outputting an alarm message as a voice signal and displaying an alarm message on a screen.

FIG. 3 is a reference diagram illustrating an interface provided for obtaining body state information of the body state information obtaining unit 100 according to an embodiment of the present invention.

As illustrated in FIGS. 2 and 3, the body state information obtaining unit 100 may obtain the body state information from the diagnostic devices configured to diagnose the body, using a wired and/or wireless interface that is currently existing or to be introduced. For example, as illustrated in FIG. 3, the interface may include a wireless and/or wired communication interface such as a wireless interface, a storage device such as a USB, Bluetooth, and RS232.

FIG. 4 is a reference diagram illustrating a process of the data processing unit 104 assigning and classifying an identifier according to an embodiment of the present invention.

As illustrated in FIGS. 2 and 4, the data processing unit 104 assigns a user identifier to the body state information obtained by the body state information obtaining unit 100 (300), assigns a device identifier (310), assigns a priority for each device (320), and assigns a time stamp (330). Then, data is classified for each device identifier or a service flow identifier is classified (340). On the other hand, the process described with reference to FIG. 4 is only an example and sequences thereof are not limited thereto. The assigned and classified identifiers for each identifier are stored and managed in a queue (350).

FIG. 5 is a diagram illustrating an identifier management structure of the data processing unit 104 according to an embodiment of the present invention.

As illustrated in FIGS. 2 and 5, the identifier structure managed by the data processing unit 104 according to the embodiment includes fields of a user identifier (User ID), a device identifier (Device ID), a service flow identifier (Flow ID), a priority identifier (Priority ID), a timestamp (Time_Stamp), a risk level (Risk_Level), a volume level (Volume_Level), a level rank (Level_Rank), and a data size (Data_Size).

FIG. 6 is a diagram illustrating a risk level management structure of the risk estimating unit 116 according to an embodiment of the present invention.

As illustrated in FIG. 6, risk level steps may be classified as C-1-R-1 (risk level rank 1), C-1-R-2 (risk level rank 2), C-1-R-3 (risk level rank 3), C-1-V-1 (volume level 1), C-1-V-2 (volume level 2), or C-1-V-3 (volume level 3), and the risks are classified in steps according to the level rank. In FIG. 6, in C-1-R-1 (risk level rank 1), Level 0 indicates risk and Level 1 indicates safety. In this case, operational notation according the number may be changed. Each level according to the risk level rank has a corresponding range value, and the range value may be managed by the function setting unit 114 or the risk information managing unit 122.

The risk estimating unit 116 may classify the body state information obtained by the body state information obtaining unit 100 into the above-described level steps. Each level step may be set by the user's setting value or a predetermined reference value according to a degree of disease of the patient. The risk level influences precision in risk estimation of the risk estimating unit 116, and the number of levels may be changed according to a requirement of precision.

FIG. 7 is a reference diagram illustrating exemplary parameter value management and setting of the function setting unit 114 according to an embodiment of the present invention.

As illustrated in FIGS. 2 and 7, the function setting unit 114 manages and sets parameter values required for performing operations of other components. That is, the function setting unit 114 manages an external interface input/output and state information, and manages and sets various identifiers. For example, the function setting unit 114 manages the device identifier, sets a classification setting value for each device identifier, and manages and sets a device identifier priority. In addition, the function setting unit 114 manages the service flow identifier, sets a classification setting value for each service flow, and manages and sets a service flow identifier priority. Furthermore, the function setting unit 114 sets the time stamp and sets queue management control. The above-described setting values are transmitted to the data processing unit 104 and used for data processing of the data processing unit 104.

Meanwhile, the function setting unit 114 manages and sets the weight assignment value. The setting value is transmitted to the weight assigning unit 118 and used to assign the weight. In addition, the function setting unit 114 manages and sets the risk information. The setting value is transmitted to the risk estimating unit 116 and used for risk estimation. Further, the function setting unit 114 manages and sets the body state information. The setting value is used to manage the body state information of the body state information managing unit 106.

FIG. 8 is a diagram illustrating a structure of a management table for each parameter generated by the risk estimating unit 116 according to an embodiment of the present invention. FIG. 9 is a diagram illustrating a structure of a management table of a blood pressure parameter which is one of parameters according to an embodiment of the present invention.

As illustrated in FIGS. 8 and 9, the management table for each parameter may include fields of a risk estimation group (RE_Group), a level rank (Level_Rank), a volume size (Volume_Size), and a risk level (Level (0 to j−1). The volume size indicates a memory capacity that may maximally swap the body state information of the patient for each parameter (service flow: for example, blood pressure, blood glucose, etc,) selected from the risk estimation group based on the time stamp. In this case, as a volume size value increases, a risk estimation probability increases. The volume size value is determined according to a degree of diseases of the patient and state information of a system internal/external memory.

In the volume size of FIGS. 8 and 9, Mc is a memory capacity counter and a value indicating a range of a swappable memory capacity. Mc is determined by a usage of an internal/external memory. The determined Mc is used as a factor determining the volume size. The volume size value (V_T (t)) may be calculated by Equation 3 or Equation 4.

$${V\_T}_{F} ( 0 ) \leq {\sum }_{L = 0}^{L = j} [ {LR}_{F} ( j - 1 ) ] \qquad [ Equation 3 ]$$

In Equation 3, the volume size value (V_T (t)) is a value of a sum of level ranks (LRs) of the service flow in any specific time (Time_Stamp)(t) zone. Equation 3 represents the volume size value (V_T (0)) when any specific time is 0 and the service flow is blood pressure (P). L denotes the risk state. For example, when the risk level rank is 5, L has a value between 0 and 5.

$${V\_T}_{F} \leq {\sum }_{L = 0}^{L = j} [ {V\_T}_{F} ( t - 1 ) ] \qquad [ Equation 4 ]$$

In Equation 4, the volume size value (V_T (t)) is a value of a sum of volume sizes of the service flow according to a time in any interval (0≦i−1≦Mc). Equation 4 represents the volume size value (V_T (t)) when the service flow is blood pressure (P). L denotes the risk state. For example, when the risk level rank is 5, L has a value between 0 and 5. Meanwhile, examples of calculating the volume size value (V_T (t)) described with reference to Equation 4 and Equation 5 are only examples for promoting understanding of the present invention, and it is apparent that various calculating methods may be used.

The RE_Group field in FIGS. 8 and 9 is a field for selecting whether the body state information for each parameter is included in order to estimate the risk using the body state information obtained in real time. The High_Low field is an information field that may determine whether the risk level is represented according to a high or low value for each parameter of the body state information of the patient. For example, blood pressure may be represented by a risk level according to hypotension or hypertension. Similarly, the blood glucose, the body temperature, and the like may be represented.

FIG. 10 is a diagram illustrating an exemplary weight range for each service flow of the weight assigning unit 118 according to an embodiment of the present invention.

As illustrated in FIGS. 2 and 10, the weight assigning unit 118 may assign different weights for each service flow, for example, blood pressure, blood glucose, an electrocardiogram, a body temperature, and a body fat percentage. The weight (W) has a value between 0 and 1. The range and value of the weight (W) may be changed and various combinations may be possible. For example, a weight of the blood glucose (S) is increased for diabetic patients, a weight of the electrocardiogram (C) is increased for heart disease patients, and a weight of the blood pressure (P) is increased for hypertension or hypotension patients, compared to other service flows. In this manner, a reference of setting the weight may be applied according to clinical results based on the patient's disease. Furthermore, the weight setting reference is set as a default value by the function setting unit 114 and may be provided to the weight assigning unit 118.

FIG. 11 is a diagram illustrating a structure of a body state information database of the body state information managing unit 106 according to an embodiment of the present invention.

As illustrated in FIGS. 2 and 11, the body state information stored in the body state information database may include information on a patient's current state value, an average value and a change amount per time range, and an average value and a change amount per period.

FIG. 12 is a diagram illustrating a structure of a regular value range database according to body state information of the body state information managing unit 106 according to an embodiment of the present invention.

As illustrated in FIGS. 2 and 12, in the body state information stored in the regular value range database, the patient's regular value range (Regular_Value) is assigned according to a patient's body type, a physical constitution, a family history, and the like.

FIG. 13 is a diagram illustrating a structure of the body state information database according to a time of the body state information managing unit 106 according to an embodiment of the present invention.

As illustrated in FIGS. 2 and 13, the body state information managing unit 106 may store and manage a measurement value (Measure_Value), which is the body state information of the patient, based on a time (Time_Stamp) set by the function setting unit 114. The time may be divided into intervals: for example, seconds, minutes, hours, day(s), week(s), and month(s).

FIG. 14 is a diagram illustrating a structure of a statistical body state information database of the body state information managing unit 106 according to an embodiment of the present invention.

As illustrated in FIGS. 2 and 14, the body state information managing unit 106 sets the risk estimation group (RE_Group) in order to apply statistical methods according to the patient's specific disease, family history, and the like. The risk estimation group is a field for selecting whether the body state information for each parameter is included in order to estimate the risk using the body state information obtained in real time and a selection may be made as Y or N. Setting the risk estimation group is used to estimate the risk in the risk estimating unit 116.

FIG. 15 is a diagram illustrating a structure of a risk information table put into a database by the risk information managing unit 122 according to an embodiment of the present invention.

As illustrated in FIGS. 2 and 15, the risk information table includes information on a risk state list, a current risk state, and an estimated risk state) with respect to a risk level rank range (2≦RLR≦j−1). The risk state list may be set according to the patient's body state information, diagnosis, and a procedure. The current risk state is represented by a degree of the current risk state according to the risk state list. The estimated risk state represents risk state information estimated according to the risk state list as a risk level step value.

In FIG. 15, the risk level rank (2≦RLR≦j−1) is a value represented by a risk state setting value set by the function setting unit 114. The risk information managing unit 122 manages the body state information of the patient according to a risk level rank value. The body state information of the patient has a unique value as a numerical value for each parameter measured in real time, and is automatically arranged in accordance with a corresponding step category whenever the risk level rank value is changed. That is, each parameter of the body state information has a measurement range value of the body state information corresponding to the risk level rank value. For example, when the risk level rank is 2, there are two steps, risk (L=0) and safe (L=1). In this case, when blood pressure is 80, it is a safe state (L=1), but when the risk level rank is 5, it may be changed to a cautious state, L=3.

FIG. 16 is a flowchart illustrating a method of controlling medical device safety of the device for controlling medical device safety 1 according to an embodiment of the present invention.

As illustrated in FIGS. 2 and 16, the body state information obtaining unit 100 obtains the body state information from the diagnostic device configured to diagnose the body. Then, the data processing unit 104 extracts an identifier from the body state information obtained by the body state information obtaining unit 100, extracts the body state information corresponding to the extracted identifier, and classifies the extracted body state information for each identifier.

Then, the weight assigning unit 118 receives a body state information service flow risk level from the function setting unit 114 (1500). In this case, when Enable is set to “1,” a weight value of the service flow identifier is loaded, the weight is assigned to a corresponding service flow (1520) according to setting of the function setting unit 114 (1510), and the assigned weight is calculated with data and then output. On the other hand, when Enable is not “1,” data is directly output.

Then, the risk estimating unit 116 generates risk estimation information from the body state information to which the weight is assigned by the weight assigning unit 118 (1530). Then, the data comparison unit 120 compares the risk estimation information generated by the risk estimating unit 116 with the body state information reference value stored in the risk information managing unit 122 (1540).

Then, the risk state determining unit 124 determines the risk state using the comparison result of the data comparison unit 120 (1560). Then, the medical device control unit 126 controls the medical device for diagnosis and treatment according to the risk state determination result of the risk state determining unit 124 (1570).

The present invention may be used in a system in which a diagnostic device and a treatment device are integrated, provide a safer procedural guideline than a current system having a structure in which a patient monitoring device, a diagnostic device, and a treatment device independently operate, and is able to be used for a one stop automation system that may diagnose and treat at the same time.

### INDUSTRIAL APPLICABILITY

The present invention may be used in a system in which a diagnostic device and a treatment device are integrated, provide a safer procedural guideline than a current system having a structure in which a patient monitoring device, a diagnostic device, and a treatment device independently operate, and is able to be used for a one stop automation system that may diagnose and treat at the same time.

[Reference Numerals]

| | |
|---|---|
| 1: device for controlling medical device safety | 2: diagnostic device |

| | |
|---|---|
| 3: medical device | 100: body state information obtaining unit |

| | |
|---|---|
| 102: signal processing unit | 104: data processing unit |

| | |
|---|---|
| 106: body state information managing unit | 108: external output control unit |

| | |
|---|---|
| 110: external output recording unit | 112: input unit |
| 114: function setting unit | 116: risk estimating unit |
| 118: weight assigning unit | 120: data comparison unit |
| 122: risk information managing unit | 124: risk state determining unit |
| 126: medical device control unit | 130: alarming unit |

1. A device for controlling medical device safety, comprising:
a body state information obtaining unit configured to obtain body state information from at least one diagnostic device for diagnosing a body;
a data processing unit configured to extract an identifier from the body state information obtained by the body state information obtaining unit, extract the body state information corresponding to the extracted identifier, and classify the extracted body state information for each identifier;
a risk estimating unit configured to generate risk estimation information according to a body state change based on the body state information extracted or classified by the data processing unit;
a risk state determining unit configured to determine a risk state using the risk estimation information of the risk estimating unit; and
a medical device control unit configured to control an external device including a medical device for diagnosis and treatment according to the risk state determination result of the risk state determining unit.

2. The device according to claim 1,
wherein the identifier includes at least one among a user identifier, a diagnostic device identifier, a time identifier, a service flow identifier, and a priority identifier according to the diagnostic device identifier or the service flow identifier.

3. The device according to claim 1,
wherein, when the body state information obtained by the body state information obtaining unit does not include the identifier, the data processing unit assigns the identifier to the obtained body state information, extracts the body state information to which the identifier is assigned, and classifies the extracted body state information for each identifier.

4. The device according to claim 1,
wherein the risk estimation information generated by the risk estimating unit has a form of a management table of the body state information for each parameter, and the management table for each parameter includes information on a risk estimation group, a level rank, a volume size, and a risk level.

5. The device according to claim 1,
wherein the risk determining unit determines a risk state using a risk estimation value that is obtained by calculating body state information having no weight or having an assigned weight for each parameter within the same time range or a predetermined time range.

6. The device according to claim 1, further comprising
a weight assigning unit configured to assign a weight to the body state information extracted or classified by the data processing unit for each service flow,
wherein the risk estimating unit generates risk estimation information from the body state information to which the weight is assigned by the weight assigning unit.

7. The device according to claim 1, further comprising
an alarming unit configured to receive the risk state determination result from the risk state determining unit through the medical device control unit and alarm the outside to the result when the risk is estimated based on the risk state determination result of the risk state determining unit.

8. The device according to claim 1, further comprising
a body state information managing unit configured to receive the body state information classified by the data processing unit, put the information into a database along with a regular value range, statistically calculate the body state information according to a body state information management setting value and a service flow, transmit a statistical result to the risk estimating unit, receive a risk estimation result from the risk estimating unit, and externally output the result.

9. The device according to claim 8,
wherein the body state information management setting value includes at least one among an identifier, a volume level, a risk level, a time, a risk estimation group, a volume size, and a level rank, and
the service flow includes at least one among a body type, a physical constitution, disease and a family history.

10. The device according to claim 1, further comprising
a risk information managing unit configured to manage risk information including a normal range and a risk state range, manage and put information on a risk state list, a current risk state, and an estimated risk state into a database within a risk level rank range according to risk estimation of the risk estimating unit.

11. The device according to claim 10, further comprising
a data comparison unit configured to compare the risk estimation information generated by the risk estimating unit with a body state information reference value stored in the risk information managing unit,
wherein the risk state determining unit determines the risk state using a comparison result of the data comparison unit.
